(19) United States
(12) Patent Application Publication
(10) Pub. No.: US 2006/0060203 A1
(43) Pub. Date: Mar. 23, 2006

(54) Drain reservoir for a tracheal or endotracheal tube

(76) Inventor: Sharon Louise Martinson, Becker, MN (US)

Correspondence Address: Jon David Schirado;Schirado Law Office, P.A., Suite 102, 6040 Main Street, Rockford, MN, 55373, US

(21) Appl. No.: 11/230,833

(22) Filed: Sep. 20, 2005

Related U.S. Application Data
(60) Provisional application No. 60/610,882, filed on Sep. 20, 2004.

(51) Int. Cl. A62B 9/06; A61M 16/00

(52) U.S. Cl. 128/207.15; 128/207.14

(57) ABSTRACT

A drain reservoir used with a tracheal or endotracheal tube to collect fluids in order to prevent the fluids from flowing back into the tube or dripping down a patient's body. A person using the drain reservoir with a tracheal or endotracheal tube can adjust the placement of the drain reservoir to isolate mucus and other fluids discharged from the tube. The drain reservoir includes an absorbent layer on the front interior, a non-liquid permeable barrier layer on the back exterior, and predetermined slits between the two layers. The layers form a reservoir shape and the tracheal or endotracheal tube is inserted through the slits. The absorbent and non-liquid permeable sides form a body cavity and sections into which the fluids travel.

### BACKGROUND OF THE INVENTION

The present invention relates to a drain reservoir, more particularly to a drain reservoir used with a tracheal or endotracheal tube. The object of the drain reservoir is to collect fluids in order to prevent the fluids from flowing back into the tube or dripping down a patient's body.

The invention came about in order to improve the utilization of tracheal and endotracheal tubes. Tracheal and endotracheal tubes are used to provide a gas passage to a patient's trachea. The patient end of the tube is inserted into the trachea and may be sealed with it by various means close to the patient end. The machine end of the tube extends out through the patient's mouth, in the case of an endotracheal tube, or through a surgically made stoma in the patient's throat, in the case of a tracheotomy tube.

In use, secretions within the trachea and bronchii tend to build up on the bore through the tracheal tube, especially where the tube remains in place for a prolonged time, such as several days. This leads to problems in that the secretions can obstruct gas passage through the tube causing difficulties in breathing for ventilation. The secretions can also provide a site for the accumulation of bacteria which, if dislodged, can be inhaled by the patient and cause bronchial infection. Furthermore, the secretions if not removed can affect the patient's hygiene.

The present invention allows the collection and removal of these fluids in an easy and economical way.

Tracheal and endotracheal tubes, as well as the methods for removing fluids from these tubes have existed for more than a century, as witnessed by U.S. Pat. No. 869,252 to Pynchon, U.S. Pat. No. 3,646,935 to Holbrook and U.S. Pat. No. 5,184,611 to Turnbull. The apparatus in the U.S. Pat. No. 869,252 reference involves an aspirator to effect the removal of fluids from cavities of the body by siphoning the fluid into an associated receiving vessel. In the U.S. Pat. No. 3,646,935 reference, a body fluid bottle utilizes antisiphoning means to prevent the backflow through the body fluid tube. The tracheal tube assembly and liner in the U.S. Pat. No. 5,184,611 reference utilizes a liner or inner tube which is inserted within the tracheal tube and which is removed and replaced by a fresh liner periodically.

While these patents provide methods for removing fluids from tracheal and endotracheal tubes, these methods do not represent an easy to use, cost-effective, and disposable method.

### BRIEF SUMMARY OF THE INVENTION

The present invention relates to a drain reservoir used with a tracheal or endotracheal tube. The disadvantages of the prior art may be overcome by providing a drain reservoir that is easy to use, cost-effective and disposable. The reservoir includes an orifice at one end, a support base at its other end and a body cavity section connecting the orifice and support base. Each section of the reservoir consists of an absorbent side and a barrier side. Disposed within the body cavity section are a plurality of slits through which the tracheal or endotracheal tube may be inserted. The tracheal or endotracheal tube may also be secured to the reservoir by attachment means. In operation, the fluid from the tracheal or endotracheal tube flows into the body of the reservoir and is collected by the absorbent surface of the reservoir as well as the support base. The reservoir is periodically removed from the tube and disposed.

It is an object of the present invention to provide a drain reservoir for use with a tracheal or endotracheal tube that isolates the mucus and other bodily fluids.

Another object of the invention is to provide a reservoir that is flexible and conformable to the patient's body.

Still another object of the invention is to create a reservoir that is disposable.

A further object of the invention is to provide a reservoir that is easy to use.

A further object of the invention is to provide a reservoir that is cost-effective.

A further object of the present invention is to provide an easy method for attaching the reservoir to the tracheal or endotracheal tube.

These together with other objects of the invention, along with the various features of novelty which characterize the invention, are pointed out with particularity in the claims annexed to and forming a part of this disclosure. For a better understanding of the invention, its operating advantages and the specific objects attained by its uses, reference should be had to the accompanying drawings and descriptive matter in which there are illustrated preferred embodiments of the invention.

### BRIEF DESCRIPTION OF THE DRAWINGS

FIG. 1 is a front perspective view of the preferred embodiment.

FIG. 2 is a side perspective view of the preferred embodiment.

FIG. 3 is a diagram for making the reservoir from a commercially available drain sponge.

### DETAILED DESCRIPTION OF THE INVENTION

In the following detailed description, certain specific terminology will be employed for the sake of clarity and a particular embodiment described in accordance with the requirements of 35 USC 112, but it is to be understood that the same is not intended to be limiting and should not be so construed inasmuch as the invention is capable of taking many forms and variations within the scope of the appended claims.

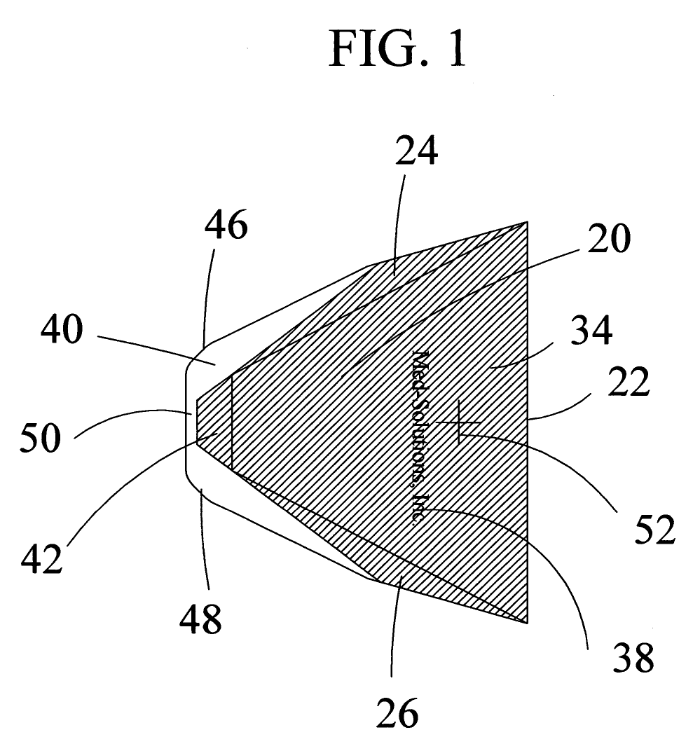

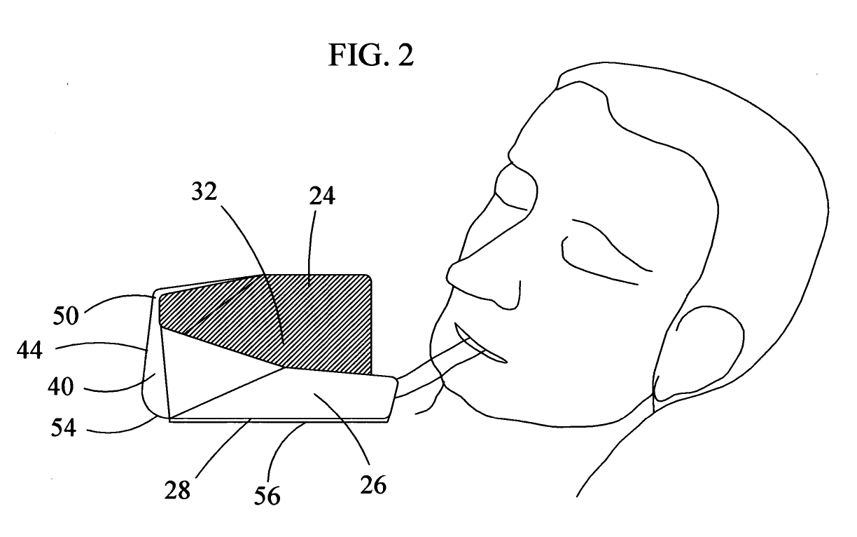

Referring to FIGS. 1-2, the present invention comprises an orifice at the top of a body cavity section, an elongated body cavity section, predetermined slits, a base section and absorbent and barrier layers.

The body cavity 20 has a top side section 34 with an orifice 22 laterally along the top. The orifice 22 allows for a wide variety of choices as to where the tracheal or endotracheal tube can be attached. The body cavity 20 has two sidewalls 24, 26, a rear wall 28 and a front wall 32.

The elongated body cavity includes a front wall 32 laminated to a rear wall 28. The front and rear walls are thin enough to provide flexibility. The distance between the side walls 24 and 26 is maintained through the use of front 32 wall and rear wall 28. The front wall 32 is made from cotton, polyester mesh or other absorbent material so as to allow the fluid to soak into the absorbent side of the front wall. The back wall 28 is made from a moisture barrier material that is non-permeable for liquids. The front wall 32 and back wall 28 may be printed with directions for use or a company logo 38 for aesthetic effect.

Located at the bottom of the elongated body cavity is the topside of the base 40. The topside of the base 40 serves as a support means for the reservoir. Disposed within said body cavity 20 is a plurality of slits 52 which may have various geometric configurations as shown in FIG. 1. The reservoir can be manufactured to have various slit patterns, or the end user can enlarge or add a slit through the body with a scissors or other cutting device, if an unusually large tracheal or endotracheal tube is used.

The base 40 can come in various shapes as long as the base design is such that the body cavity section 20, slits 52 and top section 34 can support the collection of bodily fluids. The base section 40 includes a top side 50, a bottom side 54, a proximal end 42, a distal end 44, a first side 46 and a second side 48.

The base 40 extends forward from the bottom end of the body cavity 20 to the proximal end 42 of the base 40 and back from the bottom end of the body cavity 20 to the distal end 44 of the base 40.

The proximal end 42 and the section first side 46 and second side 48 of the base 40 are fixedly attached to the side walls 24 and 26 of the body cavity. Once the reservoir is filled or otherwise ready to be removed, the base 40 and side walls 24 and 26 can easily be folded down to trap the fluid and allow for the clean removal and disposal of the reservoir.

As illustrated in FIG. 2, means can be used to secure the reservoir more firmly to the patient through an adhesive layer 56 on the rear wall 28 of the body cavity. Means can also be used to secure the orifice 22 to the tube more securely.

The preferred material from which the reservoir is made is a nonwoven material with an absorbent side and a non-permeable side. However, various other materials may be utilized as long as the interior front side is made from an absorbent material and the exterior back side is made from a moisture barrier non-liquid permeable material.

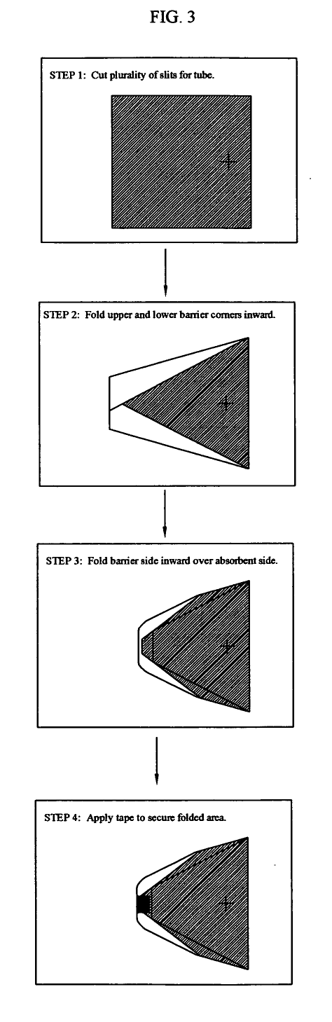

As illustrated in FIG. 3, a drain reservoir can be assembled from a commercially available drain sponge. First, make a plurality of slits in the drain sponge. Second, fold the upper and lower corners of the barrier side inward onto the absorbent side. Third, construct the base side by folding the barrier side inward over the absorbent side. Lastly, apply tape to the base to secure the folded area.

In operation of the preferred embodiment, the user takes the tracheal or endotracheal tube and inserts the tube through the slits 52 in the body cavity 20, passing the tube through the non-permeable back wall 28 and then the absorbent front wall 32. The bodily fluids travel through the tracheal or endotracheal tube and flow down the body cavity 20 towards the base 40. A portion of the bodily fluids is absorbed into the front wall 32 and side walls 24 and 26 of the body cavity as well as the absorbent top section 34 of the base. The remaining fluid is trapped within the reservoir created by non-permeable side of the body cavity and base. When removing the reservoir, the user folds down the base and side walls to trap the fluids and removes the reservoir for disposal.

The above described embodiments of the invention are intended to be an example of the present invention and alterations and modifications may be effected thereto, by those of skill in the art without departing from the scope of the invention.

What is claimed is:

1. A drain reservoir for a tracheal or endotracheal tube comprising:
a) a top orifice through which a tracheal or endotracheal tube may be inserted;
b) a body cavity extending downward from said top orifice having a plurality of slits disposed therein;
c) predetermined slits at the top of the body cavity;
d) a base supporting the body cavity; and,
e) wherein in use, bodily fluids will be absorbed into the absorbent interior of the reservoir or be trapped by the non-permeable walls of the reservoir.

2. The invention of claim 1 wherein the side walls of said body cavity are spaced by front and back walls at a distance which provides the necessary volume for sufficient fluid capture.

3. The invention of claim 2 wherein said slits are secured to and between said front and back walls.

4. The invention of claim 1 wherein the slits are made or enlarged by the end user.

5. The invention of claim 1 wherein said front wall and back wall may be printed with directions for use or a company logo.

6. The invention of claim 1 wherein said slits may have various geometric configurations.

7. The invention of claim 1 wherein said reservoir is made from a commercially available drain sponge.
